(19) United States
(12) Patent Application Publication
(10) Pub. No.: US 2023/0233347 A1
(43) Pub. Date: Jul. 27, 2023

(54) Insertion System for Implants for Treatment of Bifurcation Aneurysms

(71) Applicant: phenox GmbH, Bochum (DE)

(72) Inventors: Hans HENKES, Stuttgart (DE); Hermann MONSTADT, Bochum (DE); Ralf HANNES, Dortmund (DE); Volker TRÖSKEN, Witten (DE); Stefan ROLLA, Bochum (DE)

(73) Assignee: phenox GmbH, Bochum (DE)

(21) Appl. No.: 18/009,817

(22) PCT Filed: Jun. 14, 2021
(86) PCT No.: PCT/EP2021/065959

§ 371 Date: Dec. 12, 2022

(30) Foreign Application Priority Data
Jun. 12, 2020 (DE) 10 2020 115 614.6

(51) Int. Cl. A61F 2/954; A61F 2/90; A61F 2/97

(52) U.S. Cl. CPC A61F 2/954; A61F 2/90; A61F 2/97

(57) ABSTRACT

The invention relates to an insertion system for an implant (1) for influencing the blood flow in the region of aneurysms (22) located at vascular bifurcations. The implant (1) has two distal tubular implant portions (2) which are intended to be placed in blood vessels (21) branching off from the stem blood vessel (20) and which are connected to one another at a branching point (4). The insertion system has two sleeves (5) which are each designed to hold a distal tubular implant portion (2). The two sleeves (5) each have a distal sleeve portion (6) and the distal sleeve portions (6) each have an opening zone (7) extending in the longitudinal direction. The distal sleeve portions (6) are each adjoined proximally by a proximal portion (8), by means of which the sleeves (5) can be retracted in the proximal direction so that the opening zones (7) open and the distal tubular implant portions (2) each pass through the opening zones (7) and are released into the branching blood vessels (21). Alternatively, it is also possible to use an individual sleeve which has an opening zone for gradual release of the implant (1) or an insertion system with the implant (1) releasably attached to the outside.

The invention relates to an insertion system for an implant intended for influencing the flow of blood in the area of aneurysms that are localized at vessel branches. Aneurysms of this type are also known as bifurcation aneurysms.

Aneurysms are usually saclike or fusiform dilatations of the vessel wall and occur primarily in structurally weakened areas of the vessel wall due to the constant pressure of blood. Accordingly, the inner vessel walls of an aneurysm are thus sensitive and susceptible to injury. Rupture of an aneurysm usually leads to significant health impairment, and in the case of cerebral aneurysms, to neurological deficits and even fatalities of patients.

Aside from surgical interventions, in which, for example, the aneurysm is clamped by means of a clip, endovascular methods for the treatment of aneurysms are known in particular, with two approaches being primarily pursued. One option includes filling the aneurysm with occlusion means, especially using so-called coils (platinum spirals) for this. Coils facilitate the formation of thrombi and thus ensure occlusion of the aneurysm. On the other hand, it is known to close off the access to the aneurysm, for example the neck of an berry aneurysm, from the blood vessel side making use of stent-like implants and in this manner disconnect it from the blood flow. Both methods serve to reduce the blood flow into the aneurysm and in this way alleviate, ideally even eliminating the pressure acting on the aneurysm and thus reducing the risk of an aneurysm rupture.

When filling an aneurysm with coils it may happen that the filling of the aneurysm is inadequate, allowing blood flow into the aneurysm and in this way cause the pressure acting on its inner wall to continue. The risk of steady dilation of the aneurysm and eventually its rupture persists, albeit in an attenuated form. Moreover, this treatment method is only suitable for aneurysms having a relatively narrow neck—so-called berry aneurysms—as otherwise there is the risk that coils protrude from a wide aneurysm neck into the blood vessel where they produce clots, which may lead to occlusions in the vessel. In the worst case, a coil is completely washed out of the aneurysm and causes vessels to be occluded elsewhere. To keep the coils in place in the aneurysm sac, the aneurysm neck is often additionally covered with a special stent.

Another intravascular treatment approach focusses on so-called flow diverters. These implants are similar in appearance to stents that are used for the treatment of stenoses. However, since the purpose of these flow diverters is not to keep a vessel open, but to obstruct access to the aneurysm on the blood vessel side, their mesh width is very narrow; alternatively, implants of this kind are coated with a membrane. A disadvantage of these implants is the risk that outgoing side branches in the immediate vicinity of the aneurysm to be treated are sometimes also covered and thus closed off in the medium or long term.

Aneurysms in the area of vessel branches, in particular vessel bifurcations are a quite frequently occurring phenomenon. In the event of a weak vessel wall, the blood stream flowing through an artery and acting on the front wall in a bifurcation quickly causes a protuberance or bulge which is prone to rapidly dilate further. More often than not, such bifurcation aneurysms have a wide neck which makes therapy difficult to be performed with occlusion coils only.

Vascular implants that are suitable to bring about such a “barring” of the aneurysm entrance in the area of a vascular branching have been disclosed, for example, in the international patent applications WO 2012/113554 A1 or WO 2014/029835 A1. The aneurysm can then be rendered nonhazardous as a result of occlusion coils inserted after the implant has been placed in position. It is also possible that the implant itself separates the aneurysm sufficiently from the blood flow. For this purpose, for example, the implant may have a membrane that is placed in the area of the aneurysm neck or in front of the aneurysm neck. If considered useful or expedient, the blood flow to the aneurysm can also be reduced with filaments alone, typically wires of small diameters, to such an extent that the additional introduction of occlusion coils or other occlusion means into the aneurysm can be dispensed with.

From WO 2018/208662 A1 a flow diverter system for the treatment of bifurcation aneurysms is known, by means of which a stent-like flow diverter with a lateral opening is first inserted into the blood vessel. Subsequently, another stent-like flow diverter provided with lateral opening is passed through the opening of the first flow diverter in such a way that a Y-shape is formed overall, with the branching in the Y being located in front of the bifurcation aneurysm. In this approach, the aneurysm should be cut off from the blood flow, however, the blood flow into the branching blood vessels should remain largely unaffected.

A disadvantage of this prior art approach is that the two elements of the final flow diverter have to be inserted one after the other. This requires initially that the first flow diverter must be placed correctly before the second flow diverter has to be accurately positioned within the vasculature both relative to the aneurysm as well as relative to the first flow diverter. Due to the fact that for the treatment of bifurcation aneurysms in the intracranial region implants are as a rule inserted via the femoral artery, the treating physician must therefore have and perform very precise control over considerable distances, with the additional impediment that visualization of the implants, in particular the precise alignment of the openings inside the body, is difficult.

It is therefore the objective of the present invention to provide an insertion system for implants to influence the blood flow in the area of bifurcation aneurysms, by means of which the implant can be inserted in one step. This involves implants that have at least two distal tubular implant sections that are placed in the blood vessels branching off from a parent blood vessel. In particular, these implants may have a basic Y-shaped structure composed of two distal tubular implant sections for the branching blood vessels as well as a third, proximal tubular implant section (trunk section) for placement in the parent blood vessel.

According to a first embodiment, the objective is achieved in accordance with the invention by the provision of an insertion system for an implant for influencing blood flow in the region of aneurysms located at vessel branches, with the implant having two distal tubular implant sections which are intended to be placed in blood vessels branching off from the parent blood vessel and which are connected to one another at a branching point, wherein the insertion system comprises two sleeves, each configured to accommodate a distal tubular implant section, wherein the two sleeves each have a distal sleeve section and the distal sleeve sections each having an opening zone extending in longitudinal direction, wherein proximally adjoining each distal sleeve sections there is a proximal section via which the sleeves can be retracted in the proximal direction causing the opening zones to open and the distal tubular implant sections each to pass through the opening zones and be released in the branching blood vessels.

As provided by the present invention, the insertion system comprises a sleeve for each of the two distal tubular implant sections to be placed in one of the blood vessels branching off from the parent blood vessel. The sleeve restricts the distal implant sections and prevents them from expanding radially. At least in the distal region, both sleeves are provided with a longitudinally extending opening zone through which the distal implant sections can pass and be released when the sleeves are retracted in the proximal direction.

Alternatively, releasing the implant may be brought about by holding the sleeves in place with the aid of the proximal sections, while advancing the implant with the two distal implant sections in distal direction. The relative movement between the sleeves and the implant is of importance for the release, with the relative movement of the sleeves acting in the proximal direction, whereas the relative movement of the implant acts in the distal direction. In this context, an advancing device can be employed to exert a force on the implant in the proximal direction, either to hold it in place while the sleeves are pulled proximally or to move the implant forward in distal direction.

An opening zone in this case refers to an area of the distal sleeve sections that extends in longitudinal direction from the distal end of the distal sleeve sections proximally, typically to the beginning of the proximal sections. Preferably, the opening zones should point in the distal direction to facilitate the exit of the distal implant sections. The opening zones must reach in proximal direction to such an extent that at least a sufficiently long section of the implant can be accommodated by the sleeves and the implant is safely held by the insertion system during insertion. However, at the proximal end of the distal sleeve sections the distal implant sections protrude from the sleeves, while the sleeves are provided with proximal sections that extend further in the proximal direction.

In particular, the opening zones may consist of longitudinal slots through which the implant sections can pass when the sleeves are retracted in the proximal direction. The longitudinally extending edges of the longitudinal slots may abut or overlap to some extent, in each case ensuring that passage of the implant sections remains possible. In this context, longitudinal direction means that the opening zone must extend from the distal end of the distal sleeve sections in the proximal direction, although a course that has a certain radial component is also understood to be “in the longitudinal direction”, for example a helical course.

Instead of arranging for a longitudinal slot, it is also possible to provide another opening zone in which the longitudinal slot is only created during the release process. For example, this may be a weakening zone in which a perforation is provided that opens when the sleeve sections are pulled back, or another option can be to provide a weakening zone based on material thinning.

It is also considered expedient to provide the opening zone in such a way that the distal implant sections to be released are released from distal to proximal, which means first the two distal ends of the implant, until finally also the most proximally located areas of the distal implant sections, which are still inside the sleeve, emerge from the distal sleeve sections. This can be achieved, for example, by increasing the force to be exerted in the proximal direction for opening the opening zone from distal to proximal. For example, the resistance of a weakening zone can become stronger from distal to proximal by increasing the material thickness, using different materials or making use of a perforation with intermediate segments that increase in thickness in the proximal direction and/or openings that increase in the distal direction. In the event a longitudinal slot is provided, the overlap of the edges of the longitudinal slot may increase from distal to proximal, which also results in the distal ends of the implant being released first.

An insertion system for the placement of Y-shaped implants is also described in publication WO 2018/134097 A1. This system uses an insertion catheter that is also Y-shaped and has a branch to accommodate the distal implant sections. A continuous longitudinal slot is located at the distal end of the insertion catheter through which the implant can exit and be released when the insertion catheter is withdrawn.

An advantage of the solution proposed by the present invention over the prior art is that parts of the implant can be released one after the other, because two separate sleeves are provided for the distal implant sections placed in the branching blood vessels. The sleeves can be attended to independently of each other and are usually provided as separate parts of the insertion system. Accordingly, it is possible to first release the first distal implant section by retracting the respective sleeve proximally and subsequently the second distal implant section. In addition, it is easier to manufacture the insertion system having separate sleeves.

In order to facilitate the exit of the implant from the sleeves, the opening zones in the distal sleeve sections should open easily. For this purpose, it is expedient to manufacture the sleeves, at least in the area of the distal sleeve sections, from a flexible tubular material that yields when the sleeves are retracted allowing the implant sections to pass through.

The proximal sections of the sleeves may also be of sleeve or tubular shape. In this case, the sleeve extends preferably basically unchanged from the distal sleeve section to the proximal section, but the proximal section differs from the distal sleeve section in that it usually does not contain an opening zone, a longitudinal slot or the like. These are not required in this area because the proximal section does not contain any components of the implant. The extension of the proximal sections in proximal direction is sufficient for the treating physician to either retract the sleeves while at the same time securing the implant so that distal implant sections are released from the distal sleeve sections, or conversely, to use the proximal sections to secure the distal sleeve sections and push the implant forward in distal direction.

A sleeve- or tube-shaped design of the proximal sections is also advantageous in that in this way guidewires can be passed through the sleeves, by means of which the sleeves can be moved forward through the blood vessel system into the branching blood vessels. However, it is also conceivable in principle to design the proximal sections in different shapes, for example wire-, filament- or strip-shaped. For example, narrow strips can be cut from the tubing material used for the distal sections and used as proximal sections for the withdrawal of the sleeves. Providing a small diameter for the proximal sections and using a wire, for example, thus offers advantages in that in this way the outer diameter of the insertion system can be kept small. This is particularly beneficial due to the fact that the insertion system together with the implant is usually advanced to the target site through a microcatheter.

At the proximal end of the proximal sections a hub may be provided, for example, as is known in the prior art in the field of catheter technology. It is advisable to make use of a standard Luer or Luer-Lock connection in the area of the hub. Auxiliary means can be introduced into the interior of the proximal section via such a connection, for example guidewires. The connecting element can be made of polycarbonate, polyamide, polypropylene or other polymers, for example. As a rule, the material is stiffer than the material used for the actual sleeve sections.

Advantageously, the insertion system moreover comprises a trunk sleeve provided for accommodating a tubular trunk section of the implant. The tubular trunk section of the implant is placed in the parent blood vessel. For the purpose of releasing the trunk section of the implant independently of the sleeves for the distal implant sections, the trunk sleeve can be retracted in the proximal direction, or, otherwise, the trunk sleeve may be held in place to also release the trunk section of the implant by advancing it in the distal direction. The trunk sleeve must therefore continue in the proximal direction, with the proximal region not necessarily being sleeve-shaped, but may also consist, for example, of a wire or a similar item. However, it is important that the treating physician can effectively control the trunk sleeve. The trunk sleeve can be slid over one or more guidewires provided for this purpose. In this case, the trunk sleeve shape can continue proximally to outside the body, i.e. the trunk sleeve corresponds to an over-the-wire (OTW) system, but it is also possible for the guidewire(s) to exit further distally, similar to rapid exchange (Rx) catheters. In particular, guidewires can also be passed through the interior of the trunk section of the implant into the two distal implant sections.

By its very nature, an insertion system with a trunk sleeve to accommodate the trunk section is useful when the implant not only has two distal tubular implant sections for the branching blood vessels, but also a proximal implant section located in the parent blood vessel. Basically, the implant thus is of Y-shape with a proximal trunk section being tubular in the expanded state and arranged in the parent blood vessel as well as two distal tubular implant sections branching off from it for the branching blood vessels. However, unlike for the two sleeves for the distal implant sections, no opening zone is needed in the trunk sleeve. It is of great importance that the individual implant sections are connected in such a way that blood is allowed to flow from the parent blood vessel into the two branching blood vessels.

In principle, however, it is also a conceivable option to provide embodiments of the insertion system for the placement of implants that comprise only two distal tubular implant sections for the branching blood vessels, but no additional implant section for the parent blood vessel. Nevertheless, also of prime significance in this case is that the flow of blood from the parent blood vessel into the two branching blood vessels is ensured, i.e. in the area where the implant branches into the two distal implant sections the implant should have an opening in the proximal direction through which blood can enter from the parent blood vessel and continue to flow into the branching blood vessels.

In the event a trunk sleeve is provided, the insertion system consists of three sleeves, namely two sleeves, each of which has a distal sleeve section with an opening zone, and a trunk sleeve for the further proximally located implant section. The three sleeves can be used separately to release the implant. For example, the first distal implant section may be released first, followed by the second distal implant section, and finally the proximally located trunk section of the implant.

The trunk sleeve can also be constructed from a flexible tube material, but basically a more rigid sleeve material may also be used here, since the trunk section of the implant does not have to pass through an opening zone or slot, but is merely released by retracting the trunk sleeve.

The inventive insertion system comprising two sleeves for the distal implant sections and, if thought expedient, a trunk sleeve for the proximal implant section can be introduced into the blood vessel system through a microcatheter. Initially, the microcatheter is thus brought to the desired target site before the insertion system is advanced through the microcatheter in the distal direction. The insertion system can be pushed forward by means of two guidewires inserted into the two branching blood vessels. Subsequently, the microcatheter can first be retracted to such an extent that the implant with the surrounding sleeves is exposed before the sleeves themselves are withdrawn in proximal direction to release the implant. Finally, once the placement of the implant has been completed in the desired manner, microcatheters including the insertion system can be withdrawn in proximal direction out of the vasculature and removed.

If necessary, corrections can also be made when these operations are performed. For example, the implant including the sleeves can be retracted back into the microcatheter if it turns out that the positioning of the system requires to be optimized or other problems occur. Such withdrawal into the microcatheter is still possible even when the implant has been partially released at the distal end.

The invention relates to the described insertion system provided with two sleeves, each having a distal sleeve section for accommodating the distal implant sections, and, if considered expedient, a trunk sleeve. However, the invention also relates to a combination of insertion system and implant and/or microcatheter as well as other elements necessary or useful for the placement of the implant in front of a bifurcation aneurysm. These include, for example, guidewires.

With respect to the implant placement process, the terms “proximal” and “distal” are to be understood such that they refer to parts of the implant that point towards the attending physician (proximal), or, as the case may be, to parts that point away from the attending physician (distal). The term “axial” refers to the longitudinal axis of the implant extending from proximal to distal while the term “radial” denotes levels/planes extending vertically thereto.

In accordance with a second embodiment, the invention provides an insertion system for an implant for influencing blood flow in the region of aneurysms located at vessel branches, the implant comprising two distal tubular implant sections intended to be placed in the blood vessels branching from the parent blood vessel and which are interconnected at a branching location, wherein the insertion system comprising a sleeve having two distal sleeve arms each configured to accommodate a distal tubular implant section and being interconnected, the sleeve having a continuous opening zone in the distal direction, with the sleeve having a proximal section via which the sleeve can be retracted in the proximal direction causing the opening zone to open and thus allowing the distal tubular implant sections each to pass through the opening zone and being released in the branching blood vessels, with the opening zone being configured such that, upon release of the implant, the sleeve opens sequentially in the proximal direction from the distal ends of the distal sleeve arms.

The insertion system according to the second embodiment is based on an insertion system as described in publication WO 2018/134097 A1. Here, as already stated hereinbefore, the Y-shaped implant is released with the aid of a likewise Y-shaped insertion catheter, which has a continuous longitudinal slot at the distal end through which the implant exits upon withdrawal of the insertion catheter.

A disadvantage of this insertion system is that the implant is released across the entire width at the same time. However, it may be desirable to release the distal ends of the implant first and only gradually release the more proximally located areas. To achieve this, an insertion system is of advantage in which the opening zone is configured such that, upon release, the sleeve from the distal ends gradually opens in proximal direction. Accordingly, the distal ends of the implant, i.e. the distal ends of the implant sections intended for the branching blood vessels, can first be released from the sleeve and expand, before step by step the remaining distal implant sections are also exiting from the sleeve and adapt to the vessel inner wall. Such an approach is often advantageous for the treating physician in order to have maximum control during the release process. Before the distal implant sections are released, the sleeve restricts and prevents them from expanding radially.

To achieve this goal, the opening zone can be designed in different ways. In particular, from the outset, the opening zone may have a continuous slot, extending in the distal direction, but with the edges of the slot at least partially overlapping. This overlap should increase from the two distal ends of the sleeve arms in the proximal direction. In other words, the respective edges of the slots at the distal ends of the sleeve arms do not overlap or at most overlap slightly, but where the sleeve arms meet and are joined together, the two edges of the slot overlap significantly. Accordingly, the least force is required to release the distal implant sections at the distal ends of the sleeve arms, while this force is relatively high where the sleeve arms meet. The slot thus opens first at the distal ends of the sleeve arms and the opening continues in proximal direction so that the implant is released later in the center. This center coincides with the location that is typically placed in front of the aneurysm neck.

In accordance with another embodiment, the opening zone is provided with a weakening zone extending in the distal direction, with the force required for opening increasing in this weakening zone from distal to proximal. This zone of weakness can be achieved, for example, via a perforation in which the thickness of the webs between the openings increases from distal to proximal and/or the size of the openings increases from proximal to distal. Another possibility is to provide a thinning of material or even a variation of material in the area of the weakening zone so that the weakening zone at the distal ends of the sleeve arms tears open first when the sleeve is retracted proximally, and the opening of the weakening zone propagates in proximal direction, i.e. to the branching point of the sleeve. The characteristics of the weakness zone thus causes it to tear open as the sleeve is withdrawn from the distal ends of the sleeve arms to the location where the sleeve arms join. This also ensures a sequential release of the distal implant sections, with the more distally arranged areas being released and expanding first and the more proximal areas only slightly later.

The distal sleeve arms are connected to each other, with the connection point being located adjacent to the branching point of the distal implant sections when the implant is in the sleeve. Advantageously, the sleeve also comprises a proximal sleeve arm that forms the proximal section of the sleeve and is designed to accommodate a tubular proximal trunk section of the implant. In other words, the overall sleeve has a Y-shape, with the two distal sleeve arms accommodating the distal implant sections for the branching blood vessels, whereas the proximal sleeve arm is intended for the proximally located trunk section of the implant. The individual sleeve arms are connected to each other at a junction point and together form a hollow space for receiving the implant, which is also Y-shaped. Thus, when the proximal sleeve arm is retracted in the proximal direction, release of the proximal implant section (trunk section) also occurs, so that the implant is released as a whole. After release the trunk section of the implant widens accordingly and, in this way, secures the implant in the parent blood vessel. In principle, however, embodiments of the insertion system for the placement of implants are also conceivable that have only two distal tubular implant sections for the branching blood vessels, but no additional implant section for the parent blood vessel. Nevertheless, also of prime significance in this case is that the flow of blood from the parent blood vessel into the two branching blood vessels is ensured, i.e. in the area where the implant branches into the two distal implant sections the implant should have an opening in the proximal direction through which blood can enter from the parent blood vessel and continue to flow into the branching blood vessels.

The sleeve shape of the proximal section does not need to extend to outside the body, it is sufficient if the sleeve shape extends to the point where the proximal tubular implant section is fully accommodated within the proximal sleeve section. At this point a wire or similar element can also be connected, which allows the sleeve to be retracted in the proximal direction. At the proximal end of the proximal section a hub may be provided, for example, as it is known in the prior art in the field of catheter technology.

The sleeve, including the implant, can be transferred to the target site via two guidewires and through a microcatheter, with one of the guidewires passing through the first distal sleeve arm into the first branching blood vessel and the second guidewire passing through the second distal sleeve arm into the second branching blood vessel. The guidewires can extend through the entire sleeve, including a proximal sleeve arm, to the outside of the body (OTW configuration) or exit the sleeve proximal to the implant through a port provided for this purpose (Rx configuration).

The insertion system with implant can be advanced to the target position via a microcatheter. For release, it is possible, for example, to exert a force on the implant in the distal direction by means of an advancement device so that the sleeve can be retracted in the proximal direction, whereupon the implant exits through the opening zone. If necessary, the system consisting of the insertion system and the implant can be withdrawn even after partial release of the implant into the microcatheter has already taken place, in order to make a correction to the placement of the implant or even to abort the entire implantation procedure. An advancement device for exerting a distally acting force on the implant can be implemented in all the embodiments described in this patent application.

The insertion system can be combined with other components, in particular with the implant itself, a microcatheter, guidewires, an advancement device and the like, with such combinations also being included within the scope of the invention.

According to a third embodiment of the invention, there is provided an insertion system for an implant for influencing the flow of blood in the region of aneurysms located at vessel branches, the implant comprising two distal tubular implant sections intended to be to be placed in blood vessels branching from the parent blood vessel and which are connected to each other at a branching point, wherein the two distal tubular implant sections being in an expanded state in which they are implanted in the branching blood vessels and in a contracted state in which they are movable through the blood vessel system, wherein the insertion system has two distal shaft sections which extend through the distal tubular implant sections and which are connected with each other adjacent to the branching point, with the connection point of the distal shaft sections being proximally followed by a proximal section via which the distal shaft sections can be retracted in the proximal direction, with the distal tubular implant sections being releasably secured to the distal shaft sections so that, after the fixation has been disconnected, the distal tubular implant sections assume their expanded structure and are released in the branching blood vessels.

Unlike the first two embodiments of the invention, the third embodiment does not provide for the implant to be placed in the desired location within a sleeve, but rather provides for it to be secured to the shaft sections of the insertion system. The at least two shaft sections extend through the distal tubular implant sections intended for placement in the blood vessels branching from the parent blood vessel. At the point where the two distal implant sections are interconnected, the inner shaft sections also have a corresponding connection, with a proximal section proximately adjoining this connection of the shaft sections via which the shaft sections can be withdrawn in the proximal direction. The tubular implant sections are releasably secured to the shaft sections so that the tubular implant sections can assume their expanded structure when the fixation is undone causing said implant sections to be released in the branching blood vessels.

In other words, the implant is arranged on the insertion system when moved to the desired target site. As soon as correct placement is achieved, the fixation of the implant to the shaft sections is undone. The implant then expands and adapts to the inner vessel wall, with the two distal tubular implant sections adapting to the inner wall of the two branching blood vessels. However, in most cases another proximal tubular implant section will be provided for the parent blood vessel. As a rule, the implant is designed to be self-expanding.

The fixation points securing the distal tubular implant sections to the distal shaft sections are usefully located at least at the distal ends of the implant. As soon as the fixation points at the distal ends are unfastened, expansion of the implant begins. Additional fixation points arranged further proximally are also possible, but these are not essential because a release to occur further proximally can also be controlled via a microcatheter which surrounds the implant and the insertion system.

The shaft sections can in particular be made up of a flexible tubular material, which offers the advantage that the implant adheres well to the tubular material and an additional frictional connection is produced between the implant and the tubular material. However, it is also absolutely conceivable to use a metallic shaft, for example, on which the implant is mounted and secured.

Advantageously, the proximal section, which inter alia serves to withdraw in proximal direction the distal shaft sections located in the distal implant sections, is itself a shaft section in the sense that the proximal, tubular implant section may be arranged on this proximal shaft section. The proximal shaft section may also consist of a flexible tubing material. In this case, the implant itself typically is of Y-shape as described above, with respective internal shaft sections being provided for all three arms of the implant. Further proximally, however, the proximal section must not necessarily be designed to have a shaft form; for example, a wire or similar item which enables actuation of the shaft sections may also be fitted further proximally to a proximal shaft section on which the proximal implant section is arranged. At the proximal end of the proximal section a hub may be provided, for example, as it is known in the prior art in the field of catheter technology. It is advisable to make use of a customary Luer or Luer-Lock connection in the area of the hub. Via such a connection, auxiliary means can be introduced into the interior of the proximal section, for example guidewires or means for inducing a detachment of the connection points. These may also include means for infeeding a solvent, causing heating in an area of the shaft, or applying an electrical voltage. In the event a Luer-Lock connection is used, the connection to further elements is additionally made by screw attachment via threading provided for this purpose. The connecting element can be made of polycarbonate, polyamide, polypropylene or other polymers, for example. As a rule, the material is stiffer than the material used for the actual shaft sections.

Expediently, the shaft sections comprise an internal lumen because this allows one or more guidewires to pass through, via which the insertion system can be moved to the desired location. In particular, several guidewires can also be used, one of which, for example, is guided into the first branching vessel and the other into the second branching vessel in order to be able to advance the insertion system to the desired target site. With respect to the feedthrough of the guidewire(s), the entire proximal section can be designed to have a shaft form with an inner lumen (over-the-wire=OTW technique). However, it is also possible for the guidewires to exit via a port proximal to the implant, similar an arrangement used with Rx catheters (rapid exchange catheters).

The implant may feature a Y-shape with a third proximal tubular implant section designed to be placed in the parent blood vessel. At the branching location this third proximal implant section is connected to the other two tubular distal implant sections for the branching blood vessels, ensuring blood flow from the parent blood vessel into the branching blood vessels. The third tubular implant section is arranged on the proximal shaft section, i.e. the proximal implant section surrounds the proximal shaft section.

The third tubular implant section may have one or several releasable connection points to the proximal shaft section, including, in particular, chemically, thermally, electrolytically, or mechanically releasable connection points. Methods of releasing detachment means of this type are basically known in the state of the art.

Also the fixation points joining the tubular distal implant sections to the shaft sections may be provided in the form of chemically, thermally, electrolytically or mechanically detachable bonding connections. Detaching of the connection points can be suitably controlled by appropriately setting the respective parameters.

Chemically, thermally or electrolytically detachable connection points are understood to be connections that can be dissolved by chemical or thermal action or electrolytically by applying an electrical voltage at least to such an extent that the respective section of the implant detaches from the shaft section.

The connection points may be adhesive bonds, preferably created using a polymer adhesive. In this case, the implant is detached chemically, typically by applying a solvent that at least partially dissolves the adhesive. DMSO (dimethyl sulfoxide), for example, can be used as a solvent. A chemically detachable connection point shall also be understood as a bond that is detached by exposure to the surrounding blood alone.

There are several methods conducive to applying a solvent to the connection point. For example, a tube or other similar item can be passed along the outside of the implant and the shaft sections to dispense a solvent at the connection points. Nevertheless, it is preferred to deliver the solvent to the connection points through the interior of the shaft sections via a supply system that extends at least temporarily through the interior cavities of the shaft sections. This can be done through a tube or also through a tube provided for this purpose inside the shaft sections. The prerequisite for this is, of course, that the shaft sections, on the one hand, have an inner cavity and, on the other, sufficient permeability of the outer wall, so that the solvent applied internally can pass through the shaft sections in order to detach the adhesive bonds. Furthermore, it is also feasible to arrange one or several solvent reservoirs in the area of the connection points, which can be controlled and opened externally and, in this manner, enable the solvent to effectively act on the connections. In principle, such solvent reservoirs may also be provided on the outside of the shaft sections, provided, however, that the release of the solvent can be controlled from outside the body.

Another possible method is the electrolytic detachment of the connection points. In this case, at least partial dissolution of the connection point is achieved by applying an electrical voltage. For the most part, this is direct current, with a low current intensity (<3 mA) being sufficient. The connection point is usually made of metal and, when an electrical voltage is applied, acts as anode where oxidation and thus dissolution of the metal takes place.

Electrolytic detachment is achieved by applying voltage to the connection point making use of an electrical power source. Electrolytic detachment of implants is sufficiently known from the prior art, for example for occlusion coils for the purpose of closing off aneurysms, refer, for example, to WO 2011/147567 A1. While the connection point serves as the anode, the cathode can be positioned on the body surface, for example. Alternatively, another area of the device may also be used as cathode. It is to be understood, of course, that the connection point must be connected in an electrically conductive manner with the power source. The shaft sections themselves can serve as conductors; alternatively, a conductor can run through the interior of the shaft sections or along the outside of the implant. Since, with the cathode placed on the body surface and the corrosion current that occurs being controlled by the area of the cathode, the area of the cathode should be chosen to be significantly larger than the area of the anode. To some extent, the dissolution rate of the connection point can be controlled by selecting the cathode area relative to the anode area. The device proposed by the invention may thus also comprise a voltage source and, where applicable or appropriate, an electrode that can be placed on the surface of the body. Suitable materials for the connections to be dissolved electrolytically include stainless steel, magnesium, magnesium alloys or cobalt-chromium alloys. A particularly preferred magnesium alloy is Resoloy®, which was developed by the company MeKo from Sarstedt/Germany (cf. WO 2013/024125 A1). This is an alloy consisting of magnesium and, inter alia, of lanthanides, in particular dysprosium. Another advantage of using magnesium and magnesium alloys is that magnesium residues remaining in the body are physiologically unproblematic.

In the event of thermally detachable connections, a heat source can be brought to act on the connection points to be detached in order to release the implant. This can be accomplished from the outside by passing a heat source past the implant to allow thermal energy to be applied to the connection points. Preferably, however, the inside of the shaft sections is of hollow design, so that a heat source can also be brought through the interior of the shaft sections to the connection points to be detached. Heating of the respective shaft section inside at the position where a connection point is located on the outside ensures that this connection point is released. It is also possible to provide thermally detachable connection points that disconnect solely by the action of body temperature, in which case, however, a premature detachment must be prevented, for example by introducing and moving the insertion system and the implant to the target site through a microcatheter, with a lower temperature prevailing within the microcatheter.

After all, another option involves heating all or a single section of the shaft sections to such an extent that the connection points are dissolved causing the implant to be released.

In the event the connection point is designed to be thermally detachable, its design should ensure, on the one hand, that the implant is securely held in place on the shaft sections, but, on the other hand, excessive temperatures do not have to be applied to achieve detachment. Preferably, the at least partial dissolution of the connection point takes place at a temperature of between 40 and 80° C., in particular between 50 and 70° C. Suitable materials for the connection points, for example polymers or low-melting metals, are known from the prior art.

Another approach to securing the shaft sections to the tubular implant sections intended for placement in the branching blood vessels is to place caps on the distal ends of the distal tubular implant sections to prevent the implant sections from expanding radially. These caps can be removed in the distal direction from the distal ends, in particular can be pushed off, in order to achieve a release of the implant in this way.

In particular, the caps may be cylindrical in shape, and generally the distal end of the caps is nearly entirely closed except for a small opening for passage of a wire as described further below. The inner diameter of the caps must be such that expansion and release of the distal implant sections are prevented when the caps are in place.

In particular, removal of the caps can in particular be brought about by passing push wires at least temporarily through the inner lumen of the distal shaft sections. These push wires are designed such that advancement of the push wires in the distal direction causes the caps to slide off the distal ends of the distal implant sections. For this purpose, the push wires may, for example, have thickenings arranged on or near the distal end.

Particularly preferred is the provision of push wires which extend through openings provided for this purpose in the caps, with the push wires having thickenings distal and proximal to the openings, and with the thickening of a push wire located further proximally ensuring that the thickening rests against the interior of the cap during advancement in the distal direction and pushing the cap away from the implant when force continues to be exerted, while the distal thickenings ensure that the push wires are held in their positions and do not slip out of the openings in the caps. The thickenings should have a diameter larger than the diameter of the openings in the caps provided for the push wires, as otherwise it would be possible for the push wires to become detached from the caps. In particular, the thickenings can be spherical in shape.

To further improve the occlusion of the aneurysm, in addition to isolating the aneurysm from the flow of blood by the implant, occlusion agents may also be introduced into the aneurysm, for example coils as they are known in the prior art. Also possible is the insertion of viscous embolisates such as onyx, an ethylene vinyl alcohol copolymer, or cellulose acetate polymers.

The following comments concerning the implant apply independently of the various embodiments of the insertion system that are described in this application.

The implant itself is composed of two, preferably three, tubular implant sections, and the implant may be in an expanded state, in which it is implanted in the blood vessel, and in a contracted state, in which it is movable through the blood vessel. Two tubular implant sections for placement in the branching blood vessels are mandatory, and preferably a third, proximal implant section for placement in the parent blood vessel also exists. The implant is placed in such a way that the side of the branch point facing the distal direction is positioned in front of the aneurysm to cut it off from blood flow. This side of the branching point should therefore be as leakproof as possible. The interior of the implant, on the other hand, is unobstructed, allowing blood to flow unhindered through the parent blood vessel as well as the two branching blood vessels. Preferably, the implant sections have a circular cross-section when viewed from the proximal or distal end. In principle, however, deviations from the circular shape are also possible, for example, an oval cross-section. Where in the context of the invention reference is made to tubular implant sections, this is meant in particular with regard to the expanded state of the implant, although the respective implant sections are as a rule also tubular in the contracted state, albeit with a smaller cross-section.

With respect to the basic design of the implant, it is typically to be regarded as a flow diverter, which has been known for a long time in the prior art as a simple tubular object for sidewall aneurysms. However, the inventive flow diverter is specifically adapted for the treatment of bifurcation aneurysms, i.e., it is a bifurcation flow diverter. As a rule, it is a lattice structure consisting of wires, filaments or struts with openings arranged between them. The lattice structure of the implant can be a braided structure, i.e. consisting of individual wires or wire strands as struts, which are braided together and extend over and under each other at the points where the wires/wire strands intersect. Likewise, it can be a cut structure, in which the lattice structure is cut out of a tube of suitable diameter by means of a laser. The material is usually a metal, however plastic material may be employed as well. The elasticity of the material must be sufficient to enable contraction to take place and, moreover, upon release bring about the expansion to assume the desired diameter. Moreover, it is thought expedient to process the lattice structure by electropolishing to make it smoother and rounder and thus render it less traumatic. This also reduces the risk that germs or other impurities may adhere to the structure. The struts or wires may have a round, oval, square, rectangular or trapezoidal cross section, with the edges being advantageously rounded off in the event of a square, rectangular or trapezoidal cross section. Flat webs/wires in the form of thin strips, especially metal strips can be employed as well.

The implant to a great extent or entirely isolates the bifurcation aneurysm from the flow of blood because at least some portion of the wires comes to rest in front of the aneurysm neck. In contrast, blood flow through the parent blood vessel into the branching blood vessels is virtually unaffected. This will cause the aneurysm to undergo atrophy due to the lack of blood movement in the aneurysm causing a thrombus to form and obstruct the aneurysm.

When providing a flow diverter for bifurcations or for the treatment of bifurcation aneurysms, it is possible, for example, to attach a lateral branch for a branching blood vessel to a customary flow diverter, it being understood, of course, that the blood flow into the lateral branch must be ensured at the branching location. Alternatively, an additional branch can be arranged for the parent blood vessel, which offers advantages in that no suture or seam is created in the region of the aneurysm itself that could adversely affect the interruption of the flow of blood into the aneurysm. Moreover, two-armed implants only are also conceivable, in which the two distal tubular implant sections are placed in the branching blood vessels, whereas no section is positioned in the parent blood vessel. In this case, too, it must of course be ensured that blood can flow from the parent blood vessel into the branching blood vessels, which is why the implant has an opening facing in the direction of the parent blood vessel. Just as in the case of positioning the section for the parent blood vessel, it must be regarded as advantageous that there are no seams, sutures or other irregular braid or mesh densities present in the region of the aneurysm itself.

According to a preferred embodiment of the implant, the implant is composed of a first braided structure which is tubular in the expanded state and whose walls are consisting of individual wires braided together, wherein the first braided structure branches into a second and a third braided structure at a branching point located at a branching end, wherein the second and third braided structures are tubular in the expanded state and their walls are composed of individual wires braided together, and a portion of the wires that form the first braided structure passing into the second braided structure at the branching location, and another portion of the wires that form the first braided structure passing into the third braided structure at the branching point.

Such a bifurcation flow diverter has a braided Y-structure, that is, the implant consists of wires that are braided together by passing them over and under each other. Such a structure is particularly suitable to expand and adapt to the vessel walls after it has been released in the blood vessel. The implant comprises three interconnected tubular braided structures, with the second and third braided structures originating from the first braided structure. Of significance in this context is that the wires forming the first braided structure are at least partially transferred into the second or third braided structure. In other words, part of the wires forming the first braided structure pass into the second braided structure at the branching location, and another part merges into the third braided structure. Preferably, the implant is configured such that all wires present at the end of the first braided structure where it meets the two other braided structures (branching end) are passing into the second and third braided structures, some into the second braided structure and some into the third braided structure. It has been found that in this way a particularly good adaptation of the implant to the blood vessels is achieved and, in addition, a sufficiently high surface coverage in the area of the aneurysm neck occurs, which serves to interrupt the blood flow into the aneurysm and thus to atrophy and disable the aneurysm. The number of wires forming the first braided structure may range, for example, between 24 and 96. For example, of each two wires forming a point of intersection at the branching end of the first braided structure, one wire may pass into the second braided structure and one wire may pass into the third braided structure. Alternatively, for example, each two wires forming a first point of intersection at the branching end of the first braided structure may pass into the second braided structure, and each two wires forming a point of intersection adjacent to the first point of intersection at the branching end of the first braided structure may pass into the third braided structure.

Another approach to provide a Y-shaped bifurcation flow diverter implant is to provide first and second braided sections which are tubular when expanded and the walls of which are consisting of individual wires braided together, with the first and second braided sections each having an opening in the wall and at least the size of the opening located in the second braided section being sufficient for the first braided section to pass through, wherein the openings in the walls are created by radially displacing the wires forming the braided sections at the positions of the openings, and the first braided section is passed through the opening in the wall of the second braided section such that the opening in the wall of the first braided section faces the opening in the wall of the second braided section.

The openings in the braided sections are created at the respective positions by displacing the wires forming the braided section laterally, i.e. radially, over the circumference of the braided section. Therefore, rather than creating an opening by cutting out wires, the wires themselves are maintained unaffected without introducing a break in the wires. The wires are merely moved or shifted sideways. This is of significance in that it enables the implant in the compressed/contracted state to be reliably maneuvered to the target position. It is to be borne in mind that the advancement/feed of wires that are interrupted is often not easily possible. Moreover, the design of the implant with wires of uninterrupted configuration permits for the trouble-free expansion of the implant at the target position in the blood vessel. Since, according to the invention, the wires are not interrupted, expansion is unproblematic. Moreover, the formation of unfavorable loose wire ends that could injure vessel walls is avoided. The number of wires forming the respective braided sections advantageously amounts between 24 and 96, more preferably between 36 and 64, by which the numbers apply per braided section. The first and second braided sections may be attached to each other, e.g. sewn together, in the area of their openings.

Such an implant can be manufactured by first creating two braided sections, each provided with an opening in the sidewall. The first braid section is then passed through the opening in the second braided section and partially pulled through the interior of the second braided section. Alignment takes place in such a way that the two openings of the braided sections are facing each other. In this way, a passage is created in the transition area so that the blood inflow from the parent blood vessel can readily flow into and enter both branching blood vessels.

The first and second braided sections may be joined together at the outer end of the segment in which the two braided sections jointly extend. In this case, a different implant manufacturing method is typically applied, namely by first providing a braided structure which is tubular in the expanded state and the wall of which is composed of individual wires braided together, wherein the braided structure having a first and a second braided section which are longitudinally adjacent and arranged one behind the other, wherein both braided sections each having an opening in the wall, with at least the size of the opening in the second braided section being sufficient for the passage of the braided structure therethrough, wherein the two openings being located on opposite sides of the braided structure and the first braided section being turned over inwardly, passing through the interior of the second braided section and extending through the opening in the second braided section from the inside to the outside in such a manner that the opening in the wall of the first braided section faces the opening in the wall of the second braided section. The braided structure is thus assumed to have two braided sections, with the first braided section first being pulled through its own interior and then at least partially through the interior of the second braided section, in a way analogous to turning a sock inside out. Due to the fact that the two braided sections are connected to each other at the outer end of the segment in which the two braided sections jointly extend, i.e. are double-layered, there are no free wire ends at this point, which is advantageous in that the risk of injury to the vessel wall is reduced. Moreover, wire ends are prevented from protruding into the vessel lumen (so-called fishmouth effect) and obstructing the flow of blood.

To make sure the implant, when released in the blood vessel, automatically expands and adapts to the inner walls of the blood vessels, it is preferred to make the wires at least partially from a material having shape memory properties. Nickel-titanium alloys, for example nitinol, or ternary nickel-titanium-chromium alloys or nickel-titanium-copper alloys are particularly preferred in this context. However, other shape memory materials, for example other alloys or even shape memory polymers, are also conceivable. Materials having shape memory properties allow an implant to be imprinted with a secondary structure that it will automatically strive to adopt as soon as it is no longer hindered from expanding.

It is also possible to use so-called DFT® (drawn filled tubing) wires, i.e. wires in which the core of the wire consists of a different material than the sheath surrounding the core. It is particularly expedient to use wires having a core of a radiopaque material and a sheath of a material with shape memory properties. The radiopaque material may, for example, be platinum, a platinum-iridium alloy, or tantalum, and the material having shape memory properties is preferably a nickel-titanium alloy, as mentioned hereinbefore. DFT® wires of this type are offered by Fort Wayne Metals, for example.

Respective wires combine the advantageous characteristics of two materials. The sheath having shape memory characteristics will ensure that the implant is allowed to expand and adapt to the vessel walls, whereas the radiopaque material ensures that the implant is visible on radiographs and can thus be observed by the attending physician and positioned as needed.

The distal and proximal ends of the wires are preferably configured with a view to precluding injury to the vessel walls. For example, wires can be rounded at their ends and thus made atraumatic. Appropriate forming can be done by remelting with the help of a laser. It is also possible to join one or more wires at each end and thus create appropriate atraumatic terminations. It is, in particular, expedient to avoid pointed wire ends.

Advantageous implant coverage rates, in particular also at the branching point where the aneurysm neck is covered by implantation, are 45 to 75%, preferably 35 to 65%, for the implant in the expanded state.

Unless the context indicates otherwise, the term expanded state within the meaning of the invention is understood to denote a state which the implant assumes when it is not subject to any external constraints. Depending on the diameter of the blood vessels in which the implant is implanted, the expanded state in the vasculature may differ from the expanded state existing in the absence of external constraints because the implant may not be able to reach its completely expanded state.

In the completely expanded state, the implant sections advantageously have an outer diameter ranging between 1.5 mm and 7 mm, said diameter can be adapted to the respective target site in the blood vessel system. The overall length of the implant in the expanded state is as a rule between 5 mm and 100 mm, and in particular between 10 and 50 mm when the implant is placed such that the two distal implant sections of the Y-shaped implant for the branching blood vessels are parallel and extend in the same direction as the proximal implant section for the parent blood vessel. The wires or struts used for the implant may, for example, have a diameter or thickness ranging between 20 and 60 μm.

On the other hand, the implant may also be in a contracted or compressed state, the terms being used synonymously in the context of the present invention in the sense that the implant or an implant section has a significantly smaller radial extension in the contracted/compressed state than in the expanded state. A contracted/compressed state is assumed, for example, when the implant is brought to the target site through a catheter and positioned within the sleeves or on shaft sections.

The implant and/or the insertion system according to the invention in the form of the described shaft sections or sleeves are as a rule provided with radiopaque marker elements that facilitate the visualization and the placement at the implantation site. Such marker elements, for example, can be provided in the form of wire coils, as sleeves and as slotted tube sections that are secured to the implant and/or to the insertion system. For said marker elements, in particular platinum and platinum alloy materials are suitable, for example an alloy of platinum and iridium, as it is frequently used according to the state of the art for marking purposes and as material for occlusion coils. Other usable radiopaque metals are tantalum, gold, and tungsten. Another option is to fill the wires with a radiopaque material, as mentioned hereinbefore. It is also possible to provide the implant, in particular the wires, with a coating consisting of a radiopaque material, for example applying a gold coating. This coating can, for example, have a thickness of 1 to 6 μm. The radiopaque material coating need not be applied to the entire implant. Nevertheless, even when applying a radiopaque coating it is considered useful to arrange one or several radiopaque markers on the implant, in particular at the distal end of the implant.

The implant may also be provided with membranes that at least partially cover the implant sections, with a membrane may be used that extends over larger areas of the implant sections, or a plurality of small membranes can be provided. Such a membrane is particularly useful at the neck of the aneurysm to cut off the aneurysm from the blood flow, i.e., at the branching point in distal direction.

Covering by a membrane shall be understood to mean any type of covering, that is, the membrane may be applied to the outside of the implant sections, attached to the inside of the implant sections, or the wires/struts of the implant sections may be embedded in the membrane.

If one or more membranes are employed, it is also possible to introduce into the membranes substances that are opaque to radiation. These may be radiopaque particles as they are customarily employed as contrast medium for radio technological purposes. Such radiopaque substances are, for example, heavy metal salts such as barium sulfate or iodine compounds. A radiopaque membrane proves beneficial during placement of the implant and for localization purposes and may be used either additionally to or instead of marker elements.

Membranes may also be designed to have an antithrombogenic effect or an effect that promotes endothelial formation. Such an effect is particularly desirable where the implant is adjacent to normal vessel walls because blood flow through the vessels should not be impaired and, moreover, good anchorage of the implant in the vascular system should be achieved. Membranes can possess the desired characteristics by themselves through an appropriate choice of material, but they can also be provided with coatings that produce the desired effects.

Within the meaning of the present invention, a membrane is a thin structure having a planar surface, regardless of whether said structure is permeable, impermeable or partially permeable to liquids. However, to accomplish the objective of the aneurysm treatment, membranes are preferred that are completely or at least substantially impermeable to fluids such as blood. In addition, a membrane may also be provided with pores, particularly in the region of the aneurysm neck, through which occlusion agents can be introduced into the aneurysm. Another option is to have the membrane designed in such a way that it can be pierced with a microcatheter for the introduction of occlusion agents or even with the occlusion agents themselves.

The membranes can be made of polymer fibers or polymer films. Preferably, the membranes are produced by an electrospinning process. In this process, the wires are normally embedded in the membrane. This can be achieved by weaving or braiding fibers around the wires.

In electrospinning, fibrils or fibers are separated from a polymer solution and deposited on a substrate by applying an electric current. Said deposition causes the fibrils to agglutinate into a non-woven fabric. Usually, the fibrils have a diameter ranging between 100 and 3000 nm. Membranes created by electrospinning have a very uniform texture. The membrane is tenacious, withstands mechanical stresses, and can be pierced mechanically without an opening so created giving rise to cracks propagating from it. The thickness of the fibrils as well as the degree of porosity can be controlled by selecting process parameters as appropriate. In the context of producing the membrane and with respect to materials suitable for this purpose, special attention is drawn to publications WO 2008/049386 A1, DE 28 06 030 A1, and literature referred to therein.

In lieu of electrospinning, the membranes may also be produced by an immersion or spraying process such as spray coating. With respect to the material used for the membranes, it is important that they are not damaged by the mechanical stresses arising during implant insertion into the blood vascular system. To ensure this, the membranes should have sufficient elasticity.

The membranes can consist of a polymer material such as polytetrafluoroethylene, polyester, polyamides, polyurethanes, polyolefins or polysulfones. Especially preferred are polycarbonate urethanes (PCU). In particular, an integral connection between the membranes and the wires is desirable. Such an integral connection can be achieved by covalent bonds provided between the membranes and the wires. The formation of covalent bonds is promoted by silanization of the wires, that is, by a chemical bonding of silicon compounds, in particular silane compounds, to at least portions of the surface of the wire. On surfaces, silicon and silane compounds attach, for example, to hydroxy and carboxy groups. Basically, aside from silanization, other methods of mediating adhesion between the wires and the membranes are also conceivable.

Irrespective of relevant embodiments, the invention is particularly applicable in the neurovascular field, but applications of the apparatuses in other areas of the vascular system, such as cardiovascular and peripheral, are equally possible. Depending on the intended use, the dimensions of the components of the insertion system must be matched and adapted to each other. Thus, implants provided with tubular implant sections of small cross-section are used in particularly small blood vessels. Accordingly, the outer diameters and lumens of sleeves, sleeve sections and shaft sections should also be matched to the implants.

In the context of the invention, the term guidewire is to be understood broadly and the guidewire may be both solid in cross-section and equipped with an internal cavity/lumen.

Aside from the insertion systems themselves, the invention also relates to combinations comprising insertion systems, implants, microcatheters and/or other aids such as guidewires and advancement devices. Within the scope of the invention, two guidewires in particular can be used for placing the implant in the two branching blood vessels. Moreover, the invention relates to the use of the insertion system for the placement of implants with a view to treating arteriovenous malformations, in particular (bifurcation) aneurysms. In addition, the invention also relates to a relevant method. All statements made with reference to the insertion system itself also apply in an analogous manner to the combination with further components, to the use and to the method.

Further elucidation of the invention is provided by way of examples through the enclosed figures. It is to be noted that the figures show preferred embodiment variants of the invention, with the invention itself not being limited thereto. To the extent it is technically expedient, the invention comprises, in particular, any optional combinations of the technical features that are stated in the claims or in the description as being relevant to the invention. All statements made with respect to one embodiment of the invention apply in the same way to the other embodiments of the invention, unless the context indicates otherwise.

Clarification of the invention is provided by the following figures where

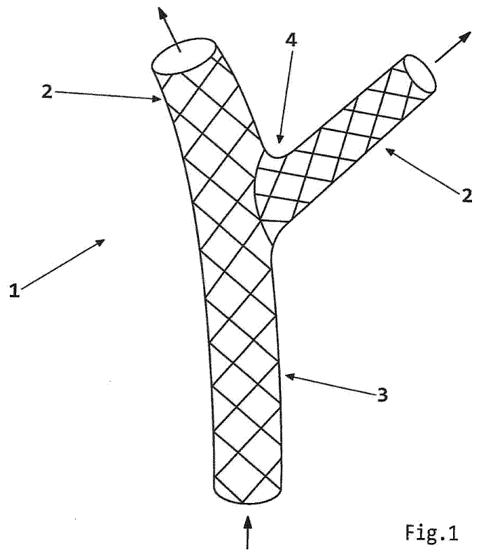

FIG. 1 shows a Y-shaped implant with two distal and one proximal tubular implant section;

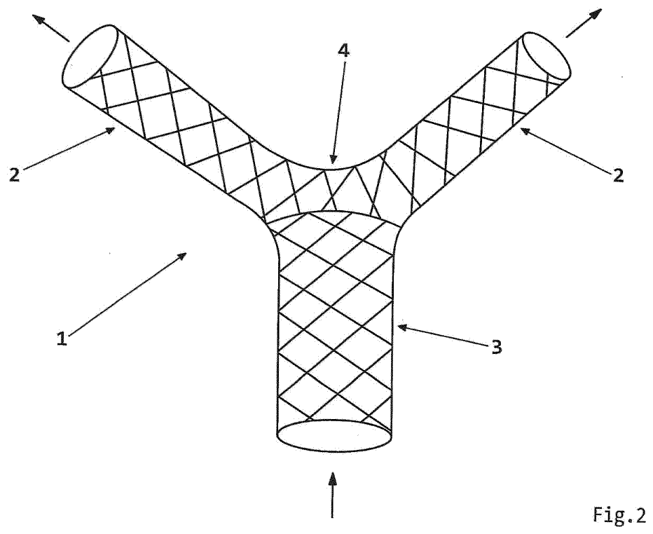

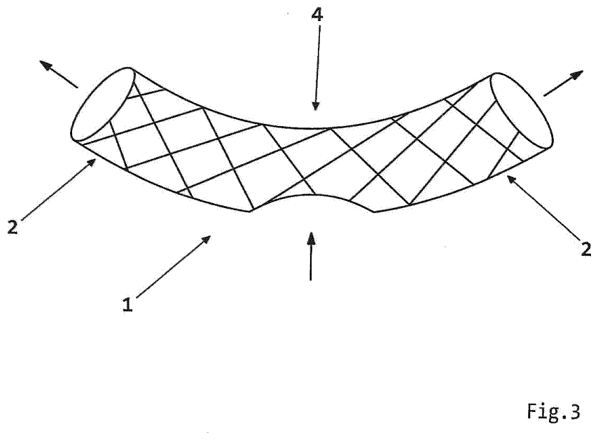

FIG. 2 shows another Y-shaped implant with two distal and one proximal tubular implant section;

FIG. 3 illustrates an implant having two distal tubular implant sections without a proximal implant section;

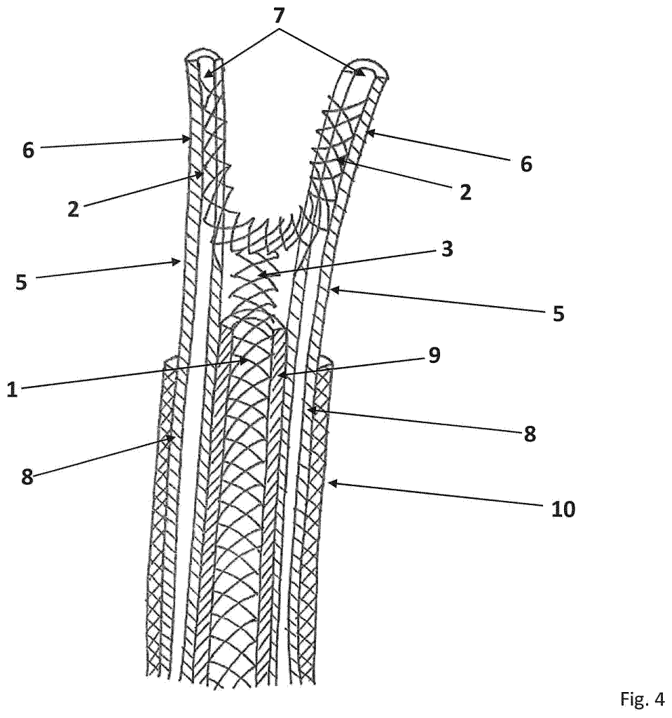

FIG. 4 shows an inventive insertion system according to a first embodiment;

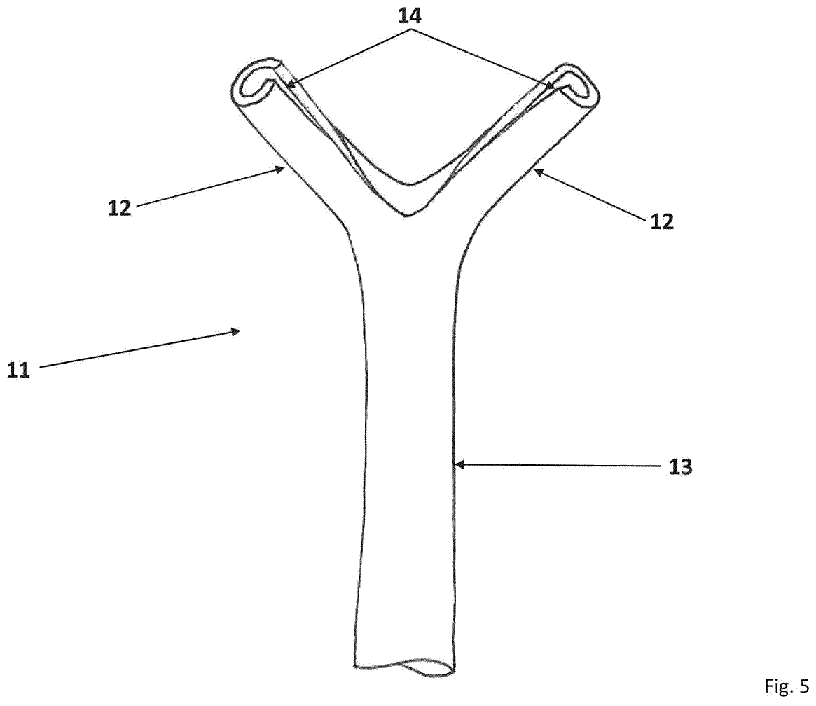

FIG. 5 shows an inventive insertion system according to a second embodiment;

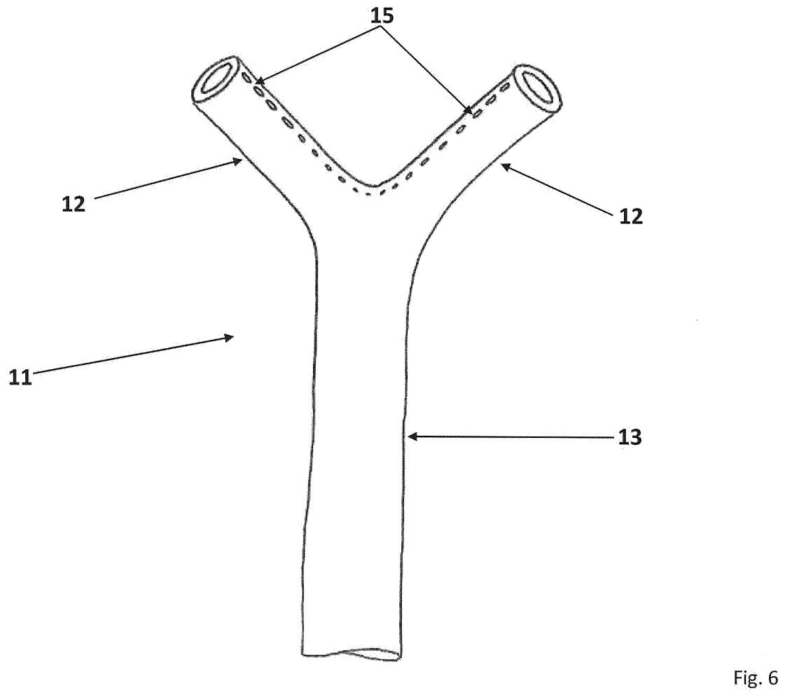

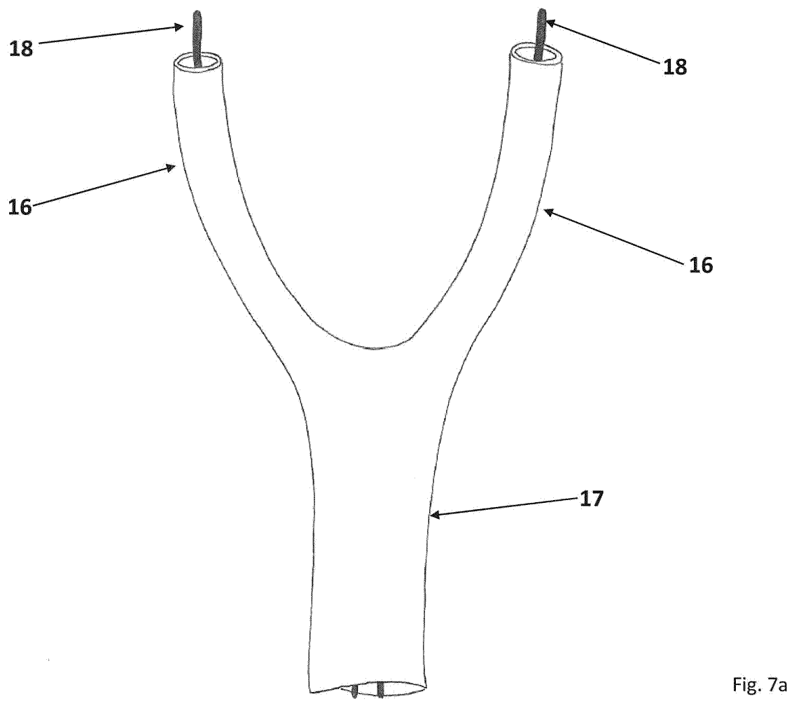

FIG. 6 is another variant of the second embodiment of the insertion system;

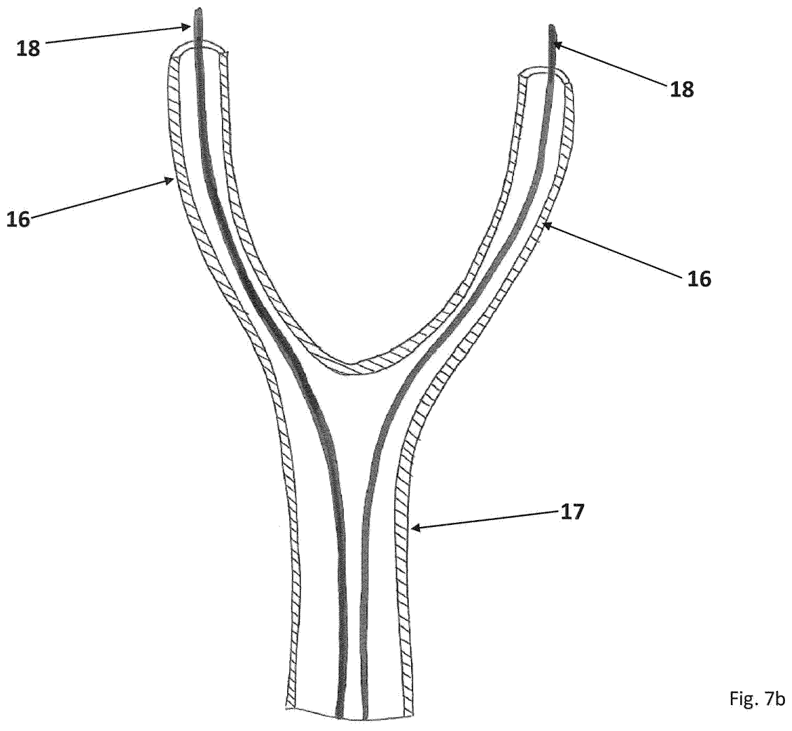

FIG. 7a illustrates an inventive insertion system according to a third embodiment;

FIG. 7b is a sectional view of the insertion system shown in FIG. 7a;

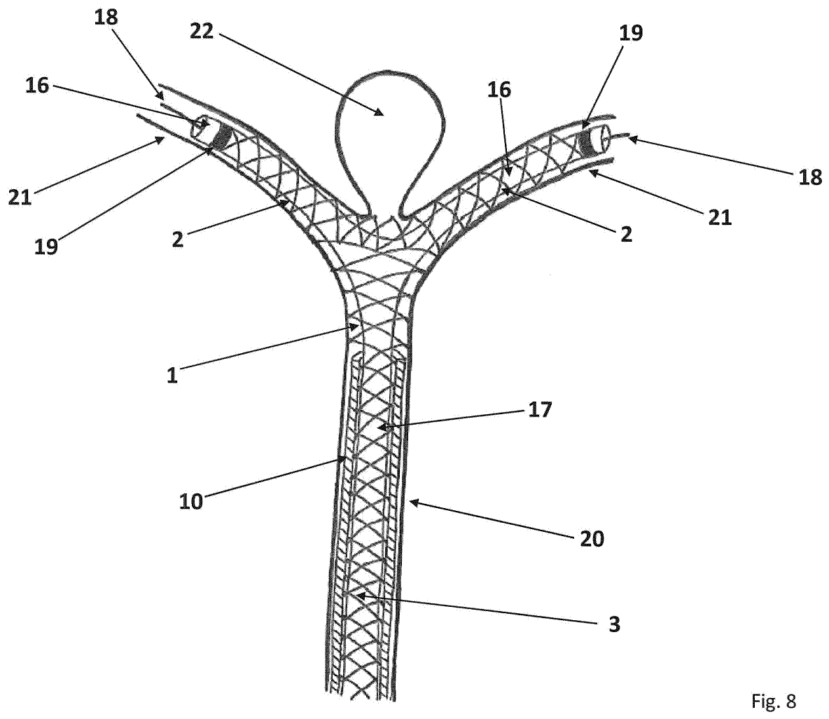

FIG. 8 shows the insertion system according to FIG. 7a,b with the implant placed in front of an aneurysm;

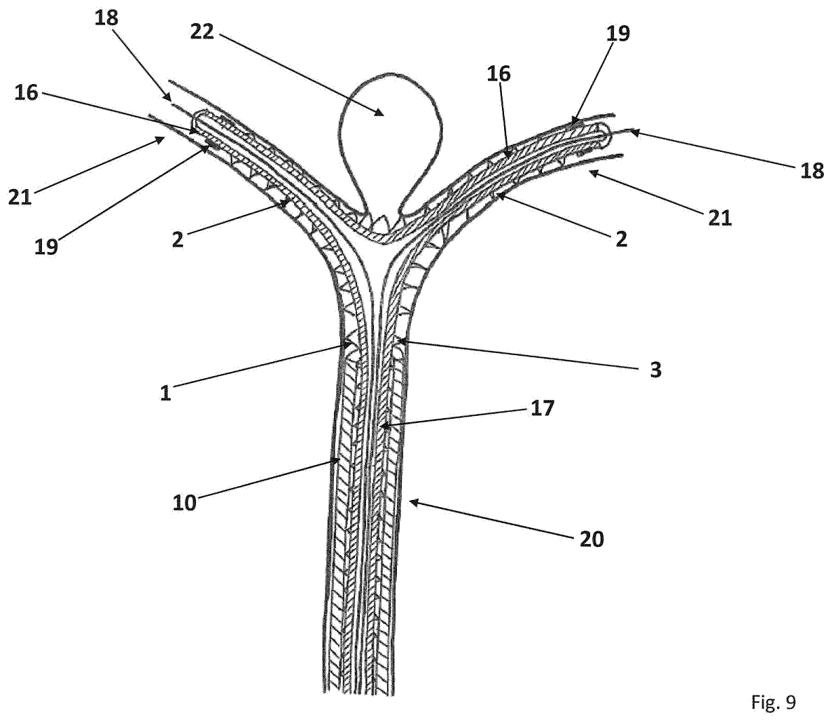

FIG. 9 is a sectional view of the insertion system with implant placed according to FIG. 8 and

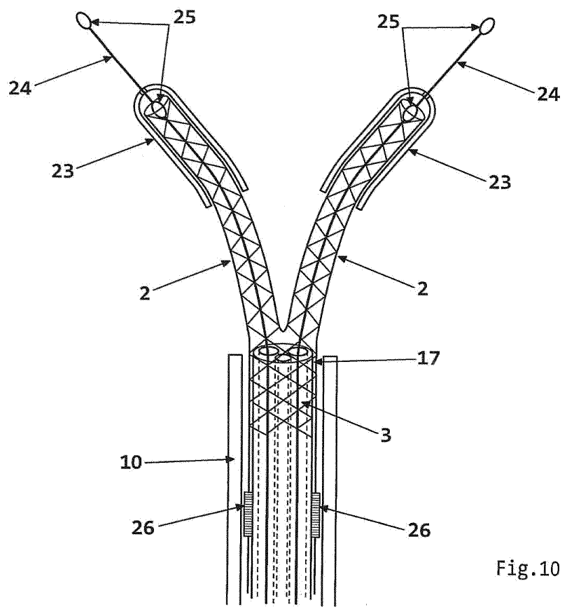

FIG. 10 is another variant of the third embodiment of the insertion system.

FIG. 1 shows an implant 1, i.e. a bifurcation flow diverter, as it can be introduced into the vascular system and placed in front of a bifurcation aneurysm with the aid of the insertion system proposed by the present invention. Implant 1 has three tubular implant sections 2, 3, of which the two distal implant sections 2 are positioned in the branching blood vessels, whereas the proximal trunk section 3 is anchored in the parent blood vessel. The aneurysm is located distal to branch point 4 between the two distal implant sections 2. Blood flow from the parent blood vessel into the two branching blood vessels has been indicated by arrows.

The implant 1 illustrated here is essentially a round braiding that merges from the proximal trunk section 3 into one of the two distal implant sections 2, with an additional branching arm being attached as an additional distal implant section 2. It is, of course, mandatory that the configuration of the connection between the individual implant sections 2, 3 makes sure the flow of blood also takes place into the second, attached distal implant section 2. Moreover, there should be good coverage of the aneurysm by a tight braid of the wires or struts used.

FIG. 2 shows an implant 1 which is essentially similar to the implant depicted in FIG. 1, but in this case a continuous tubular structure has been provided for the two branching blood vessels and a lateral branch has been attached as proximal trunk section 3. Here too, of course, the passage of the flow of blood from the parent blood vessel into the two branching blood vessels must be ensured in the direction indicated by the arrows. A certain advantage over the variant shown in FIG. 1 can be seen in the fact that in the area distal to the branching point 4 no seams or irregular mesh or braid densities are to be expected, which could under some circumstances cause too high a permeability in the direction of the aneurysm.

FIG. 3 is the representation of another implant 1 that does not have a proximal trunk section, but consists only of two distal implant sections 2. In this case as well, it is also mandatory that the aneurysm is covered distal to the branch point 4; and, additionally, the flow of blood from the parent blood vessel into the two distal implant sections 2 must be ensured, which is why an opening is arranged in the implant structure in the proximal direction through which blood can flow as symbolized by the arrows.

FIG. 4 shows a first embodiment of the insertion system according to the invention together with the implant 1 to be placed in position. The insertion system is provided with two sleeves 5, the distal sleeve sections 6 of which accommodate the distal implant sections 2. In the proximal direction, proximal sections 8 respectively join the distal sleeve sections 6 so that the sleeves 5 can be retracted as a whole in the proximal direction to release the implant 1. The proximal sections 8 can also be designed to be sleeve-shaped or tubular. A third sleeve is provided as a trunk sleeve 9 and surrounds the proximal trunk section 3 of the implant 1. The trunk sleeve 9 can also be retracted in the proximal direction for the purpose of releasing the proximal trunk section 3.

In order to allow the implant 1 and the distal implant sections 2 to emerge from the distal sleeve sections 6, these are provided with opening zones 7. These are arranged in the form of longitudinal slots that run along the sleeves 5 over a certain area in the longitudinal direction. In this case, the opening zones 7 extend from the distal end of the distal sleeve sections 6 in the proximal direction to such an extent that the distal implant sections 2 can pass through completely when the sleeves 5 are withdrawn in the proximal direction. Proximal to the opening zones 7, on the other hand, the sleeves 5 can essentially be designed as a simple sleeve or tube, since the passage of the implant 1 is no longer an issue in this area.

The insertion system can be brought to the intended position by a microcatheter 10. As soon as this position is reached, the microcatheter 10 can initially be retracted at least far enough in the proximal direction to expose the insertion system with the implant 1 inside. Following this, the sleeves 5 can be retracted simultaneously or one after the other in proximal direction to release the two distal implant sections 2, whereupon they expand and come in contact with the inner vessel wall of the branching blood vessels. As a rule, the proximal implant section 3 is released last by withdrawing the trunk sleeve 9 of the insertion system in the proximal direction.

In FIG. 5 a first variant of an insertion system is depicted according to the second embodiment, with the implant not shown. The insertion system is provided with a sleeve 11 that has two distal sleeve arms 12. The distal sleeve arms 12 are intended to accommodate the distal implant sections 2. Connected to the two distal sleeve arms 12 is the proximal sleeve arm 13, in which the proximal trunk section 3 of the implant is positioned.

The two distal sleeve arms 12 are provided with a slot 14 extending from the distal end of the first distal sleeve arm 12 to the distal end of the second distal sleeve arm 12. The slot 14 is configured such that the edges along the slot 14 do not overlap at the distal ends of the distal sleeve arms 12, with an overlap of the slot edges existing however in the area where the proximal sleeve arm 13 branches into the two distal sleeve arms 12. The effect of this is that when sleeve 11 is withdrawn, the implant initially emerges from the slot 14 at the distal end of the distal implant sections 2 and begins to expand, whereas the regions of the implant 1 located farther proximal only emerge from the sleeve 11 a little bit later. Accordingly, the distally located areas of implant 1 adapt to the inner wall of the blood vessel first and only at a slightly later time do the more proximally located areas contact the wall, which is advantageous with regard to a controlled release of implant 1.

FIG. 6 shows an insertion system which is essentially identical to that indicated in FIG. 5, but in this case the opening zone is not provided in the form of a slot 14, but in the form of a perforation 15. The size of the openings arranged in the perforation 15 in this case increases from proximal to distal, i.e. in the region of the branching between the two sleeve arms 12 the size of the openings is relatively small, whereas at the distal ends of the distal sleeve arms 12 it is significantly larger. The perforation 15 thus tears open first at the distal end of the distal sleeve arms 12 when the sleeve 11 is withdrawn in the proximal direction, so that in this location as well the implant emerges from the sleeve 11 first and can then expand. Only slightly later is the implant also released further proximally, which is due to the fact that tearing open the perforation 15 requires more force to be applied here. Therefore, the implant is released gradually from distal to proximal.

In FIG. 7a the insertion system according to a third embodiment is shown in which the implant to be inserted is arranged on the outside. The implant itself is not shown here. This is a Y-shaped inner shaft system that has a proximal shaft section 17 branching into two distal shaft sections 16. The insertion system is brought to the intended position with the aid of two guidewires 18, one of the guidewires 18 being inserted into the first branching blood vessel and the other guidewire 18 being inserted into the second branching blood vessel in order to be able to subsequently move the insertion system to the desired position via the guidewires 18, with the two distal shaft sections 16 being guided at the same time into the respective branching blood vessels.

FIG. 7b is a sectional view of the insertion system shown in FIG. 7a, i.e. it can be seen how guidewires 18 first extend in parallel through the proximal shaft section 17 and then branch into the two distal shaft sections 16.

In FIG. 8 the system from FIG. 7 is illustrated with implant 1 applied in the blood vessel system. The insertion system was placed in front of an aneurysm 22, with the two distal shaft sections 16 being placed in the branching blood vessels 21 and the proximal shaft section 17 being placed in the parent blood vessel 20. Implant 1 again features two distal implant sections 2 and the proximal trunk section of the implant 3. At the two distal ends of the distal implant sections 2, the implant 1 is connected to the distal shaft sections 16 by means of fixation points 19. The fixation points 19 may be, for example, chemically or electrolytically dissolvable connecting points, i.e., detachment of the implant 1 from the distal shaft sections 16 causes the implant 1 to expand and be released from the shaft sections. The shaft sections 16, 17 forming part of the insertion system can subsequently be withdrawn in a proximal direction while the implant 1 remains in the desired position in the blood vessel system in front of the aneurysm neck.

The insertion system including implant 1 is brought to the desired position by a microcatheter 10. Release of the implant 1 in the region of the proximal implant trunk section 3 typically occurs by retraction of the microcatheter 10 in the proximal direction.

FIG. 9 shows the insertion system with implant 1 of FIG. 8 in sectional view; it can be seen how the guidewires 18 pass through the interior of the trunk sections 16, 17, while the distal implant sections 2 are arranged on the two distal trunk sections 16 and the proximal implant trunk section 3 is seated on the proximal trunk section 17.

Finally, FIG. 10 shows an alternative form for securing the distal implant sections 2 to the insertion system, with the distal shaft sections having largely been omitted here. Caps 23 are placed over the distal ends of each of the distal implant sections 2 to prevent the distal implant sections 2 from expanding while the caps 23 are in position on the distal implant sections 2. To enable the caps to be removed from the implant sections 2, push wires 24 are advanced in the distal direction. The push wires 24 extend through small openings at the distal ends of the caps 23 and are provided with thickenings 25, so that as the push wires 24 are advanced distally, the thickenings 25 located further proximally will move the caps 23 forward and in this way remove them from the implant sections 2. The farther distally located thickenings 25 are provided to prevent the push wires 24 from becoming disconnected from the caps 23. Moreover, radiopaque markers 26 are provided on the proximal shaft section 17 to allow the implantation process to be visualized.

1. An insertion system for an implant (1) for influencing the flow of blood in the region of aneurysms (22) located at vessel branches, with the implant (1) having two distal tubular implant sections (2) which are intended to be placed in blood vessels (21) branching off from the parent blood vessel (20) and which are connected to one another at a branching point (4),
wherein
the insertion system is provided with two sleeves (5), each configured to accommodate a distal tubular implant section (2), wherein the two sleeves (5) each being provided with a distal sleeve section (6) and the distal sleeve sections (6) each having a longitudinally extending opening zone (7), wherein proximally adjoining each of the distal sleeve sections (6) there is a proximal section (8) via which the sleeves (5) can be withdrawn in the proximal direction so that the opening zones (7) open and each of the distal tubular implant sections (2) passes through the opening zones (7) and are released in the branching blood vessels (21).

2. An insertion system according to claim 1, wherein the opening zones (7) are provided in the form of longitudinal slots.

3. An insertion system according to claim 1, wherein the sleeves (5), at least in the region of the distal sleeve sections (6), are composed of a flexible tube material.

4. An insertion system according to claim 1, wherein the proximal sections (8) are designed to be sleeve-shaped or tubular.

5. An insertion system according to claim 1, wherein the proximal sections (8) are designed to be in the form of wires, filaments or strips.

6. An insertion system according to claim 1, wherein, the insertion system comprises a trunk sleeve (9) provided for accommodating a tubular trunk section (3) of the implant (1), the trunk sleeve (9) being withdrawable in proximal direction for releasing the trunk section (3).

7. An insertion system according to claim 6, wherein the trunk sleeve (9) is made of a flexible tube material.

8. An insertion system for an implant (1) for influencing the flow of blood in the region of aneurysms (22) located at vessel branches, wherein said implant (1) comprising two distal tubular implant sections (2) which are intended to be placed in blood vessels (21) branching off from the parent blood vessel (20) and which are connected to each other at a branching point (4), wherein the insertion system being provided with a sleeve (11) having two distal sleeve arms (12) which are each configured to accommodate a distal tubular implant section (2) and which are interconnected, the sleeve (11) having a continuous opening zone (14, 15) in the distal direction and with the sleeve (11) having a proximal section (13) via which the sleeve (11) can be retracted in the proximal direction so that the opening zone (14, 15) opens and each of the distal tubular implant sections (2) pass through the opening zone (14, 15) and are released in the branching blood vessels (21),
wherein
the opening zone (14, 15) is designed such that the sleeve (11) opens sequentially from the distal ends of the distal sleeve arms (12) in the proximal direction when the implant (1) is released.

9. An insertion system according to claim 8, wherein the opening zone (14, 15) comprises a continuous slot (14) pointing in the distal direction, wherein the edges of the slot (14) at least partially overlap in such a way that the overlap increases from distal to proximal.

10. An insertion system according to claim 8, wherein the opening zone (14, 15) comprises a weakening zone facing in the distal direction, with said weakening zone being designed such that the force required for opening increases from distal to proximal.

11. An insertion system according to claim 10, wherein the weakening zone is a perforation (15).

12. An insertion system according to claim 8, wherein the proximal section (13) is a proximal sleeve arm provided for accommodating a tubular trunk section (3) of the implant (1).

13-27. (canceled)
